(19) United States
(12) Patent Application Publication
(10) Pub. No.: US 2010/0210925 A1
(43) Pub. Date: Aug. 19, 2010

(54) Optical Apparatus for Measuring a Physiological Property of a Body Part and Method Therefore

(76) Inventors: Richard Holley, Berkshire (GB); Austen Hearn, Berkshire (GB)

Correspondence Address: BRACEWELL & GIULIANI LLP, P.O. BOX 61389, HOUSTON, TX, 77208-1389, US

(21) Appl. No.: 12/671,229

(22) PCT Filed: Jul. 29, 2008
(86) PCT No.: PCT/GB2008/050635

§ 371 Date: Apr. 29, 2010

(30) Foreign Application Priority Data
Jul. 30, 2007 (GB) 0714738.2

(51) Int. Cl. A61B 5/1455

(52) U.S. Cl. 600/310

(57) ABSTRACT

An optical measurement apparatus comprises an optical system (100). The optical system (100) includes a source (102) and a detector (112). The source, when in use, emits a probe beam (116) and the optical system is arranged to direct the probe beam (116) to a location to be measured (114). The detector (112) is arranged to receive a reflected beam (120) from the location to be measured (114).The optical system (100) has a receiving numerical aperture associated therewith and is also arranged so that the probe beam (116) has a probe cone angle corresponding to a probe numerical aperture. The receiving numerical aperture is greater than the probe numerical aperture. Alternatively or additionally, the optical system (100) is arranged to apply a magnification ratio between the detector (112) and the location to be measured (114) so as to minimise deviation of the reflected beam from a receiving axis (119) of the optical system (100).

The present invention relates to an optical measurement apparatus of the type that, for example, is used to measure a physiological property of a body part, such as an eye. The present invention also relates to a method of optically measuring a location to be measured, the method being of the type that, for example, is used to measure a physiological property of a body part, such as an eye.

Diabetes is a major and rapidly growing problem with over 230 million people suffering from the disorder worldwide. In addition, studies have shown that the incidence of juvenile-onset, insulin-dependent diabetes has doubled over the last 15 years. There has also been a five fold increase in the number of children under the age of 5 suffering from diabetes in just 20 years.

The symptoms associated with diabetes can be severe. If the blood glucose level is not suitably controlled by the patient, the physical damage which may be caused includes blindness, heart disease and gangrene. As such, the mortality rate for people with diabetes is significantly higher than the rate for the average person.

A person's blood glucose concentration varies over a relatively short timescale, due to a number of factors, such as the length of time since the patient's last meal, the type of food ingested, the amount of exercise taken, and whether or not the patient is otherwise ill. As a result, people with diabetes usually need to test their glucose levels many times a day, in order to monitor and control their condition. The actual testing regime varies between patients and is individually prescribed by the doctor or diabetes educator of the patient.

The primary method used for testing blood glucose concentration involves the taking of a blood sample, which is then analysed. In this test, a patient's finger or arm is pricked with a small needle and the resulting drop of blood is placed on a test strip, for analysis in a hand-held meter. If the glucose concentration reading is above an acceptable level, insulin must be injected to bring the glucose concentration back within an acceptable range.

Due to the frequency of testing required to monitor the blood glucose concentration, the patient is normally expected to perform the tests throughout the day, drawing and analysing the blood sample himself. There are a number of problems experienced by patients with the above procedure. Firstly, the technique is invasive and therefore carries the risk of infection. Secondly, continual pricking of the fingers causes hard skin. Thirdly, the process is clearly not pain-free. Finally, there is a large, ongoing consumables cost associated with this method. As a result of these and other problems, certain sectors of the diabetic population do not test themselves as often as required. This is particularly the case for the elderly, who tend to lack the fine motor skills required; teenagers, who tend to find the whole procedure socially embarrassing; and children, who tend not to accept the discomfort associated with the process.

A number of non-invasive blood glucose concentration measuring techniques have been proposed to overcome these problems. In general these have been designed to work by making a measurement through the skin but the variability in the skin's characteristics have led to inaccurate results.

More recently the eye has been proposed as a better measurement location. Possible techniques for measuring glucose in the eye include spectroscopy on the conjunctiva (e.g. U.S. Pat. No. 6,975,892), psychophysical measurements on the fundus (e.g. U.S. Pat. No. 6,895,264), a contact lens or other implantable device that absorbs glucose (e.g. U.S. Pat. No. 6,980,842 or U.S. 2006/0166350) or a measurement of the ocular refractive correction (e.g. U.S. Pat. No. 6,442,410).

One particular approach which has been suggested involves measuring the glucose concentration of the aqueous humour in the anterior chamber of the eye, since, while varying between individuals, there is a close correlation between this concentration and the blood glucose concentration. Measurement of the glucose concentration of the aqueous humour may be achieved by various means; for example, by polarimetry (e.g., U.S. Pat. No. 5,896.198); by Raman techniques (e.g., WO A 00/02479); by fluorescence photometry (e.g., WO 2005/120334); by spectrometry (e.g., U.S. Pat. No. 5,969,815); by fluorescence spectroscopy (e.g., WO 02/087429) or by reflectometry (e.g., U.S. Pat. No. 6,236,089).

A desirable alternative approach to measuring the glucose concentration in the aqueous humour involves measuring the refractive index of the aqueous humour, since there is a strong correlation between the refractive index and the glucose concentration. In this respect, U.S. Pat. No. 3,963,019, U.S. Pat. No. 6,152,875, WO 03/025562, WO 05/044099 and WO 05/058152 describe various techniques associated with measurement of the refractive index of the aqueous humour.

In addition there are many other measurements that require an instrument to be aligned to the eye of a patient. In one example, it is necessary to measure the thickness or shape of the cornea in order to make Laser-Assisted in Situ Keratomileusis (LASIK) surgery safer (for example as described in U.S. Pat. No. 6,585,723 and U.S. 2004/0080759). Alignment to the eye of a patient is also required during the measurement of ophthalmic characteristics, such as Central Corneal Thickness (CCT), Anterior Chamber Depth (ACD), corneal curvature and/or axial length of the eye.

In all of the above cases, the measurement fidelity is compromised by variations in alignment between the meter and the patient's eye. In addition, for a successful personal use meter, it is important that the patient is able to use the meter by themselves, and align to the meter by themselves, without any clinician involvement. In the case of a meter used in a clinical environment, it is important that the clinician knows that a valid measurement has been made.

Furthermore, at times it is not always possible to achieve good alignment to the eye when measuring glucose levels and other parameters of the eye non-invasively.

According to a first aspect of the present invention, there is provided an optical measurement apparatus comprising: an optical system comprising: a source arranged to emit, when in use, a probe beam, the optical system being arranged to direct, when in use, the probe beam to a location to be measured; and a detector arranged to receive, when in use, a reflected beam from the location to be measured; wherein the optical system has a receiving numerical aperture associated therewith, the optical system being further arranged so that, when in use, the reflected beam has a cone angle corresponding to a reflected beam numerical aperture; and the receiving numerical aperture is greater than the reflected beam numerical aperture.

The receiving numerical aperture may be provided so as to prevent, when in use, vignetting of the reflected beam, the reflected beam numerical aperture being substantially the same as a probe numerical aperture corresponding to a probe cone angle of the probe beam.

A probe numerical aperture corresponding to a probe cone angle of the probe beam may be set so as to prevent, when in use, vignetting of the reflected beam as a result of the receiving numerical aperture.

The optical system may have a receiving axis associated with the receiving numerical aperture; and the receiving numerical aperture may be arranged to permit acceptance of the reflected beam when deviated from the receiving axis by up to a predetermined maximum off-axis reflection angle with respect to the receiving axis.

The optical system may be arranged to constrain a probe numerical aperture corresponding to a probe cone angle of the probe beam so that, when in use, the reflected beam may be incident within an acceptance angle of the optical system corresponding to the receiving numerical aperture.

The optical system may comprise an optical path from the source to the detector via the location to be measured. The optical system may further comprise an optical element disposed in the optical path.

The optical element may be a refractive optical element. The optical element may be a lens. The optical element may be a diffractive optical element. The optical element may be reflective.

The optical element may be an aperture.

The optical element may provide the receiving numerical aperture.

The probe numerical aperture may be dynamically adaptable.

The apparatus may further comprise a receiving axis, and wherein the optical system may be arranged to apply, when in use, a magnification ratio between the detector and the location to be measured so as to minimise deviation of the reflected beam from the receiving axis.

A waveguide arrangement may comprise at least part of the optical system. The waveguide arrangement may be a fibre-optic arrangement.

According to a second aspect of the present invention, there is provided a confocal measurement apparatus comprising the optical measurement apparatus as set forth above in relation to the first aspect of the invention.

According to a third aspect of the present invention, there is provided a method of optically measuring a location to be measured, the method comprising: emitting a probe beam; directing the probe beam to the location to be measured; providing an optical system capable of receiving a reflected beam from the location to be measured, the optical system having a receiving numerical aperture associated therewith; wherein the reflected beam has a cone angle corresponding to a reflected beam numerical aperture; and the receiving numerical aperture is greater than the reflected beam numerical aperture.

According to a fourth aspect of the present invention, there is provided an optical measurement apparatus comprising: an optical system comprising: a source arranged to emit, when in use, a probe beam, the optical system being arranged to direct, when in use, the probe beam to a location to be measured; a detector arranged to receive, when in use, a reflected beam from the location to be measured; and a receiving axis; wherein the optical system is arranged to apply, when in use, a magnification ratio between the detector and the location to be measured so as to minimise, when in use, deviation of the reflected beam from the receiving axis.

The deviation of the reflected beam may be due to misalignment of location to be measured with the receiving axis.

The receiving axis may be associated with a substantially ideal optical receive path.

The optical system may have an object plane and an image plane, the magnification ratio being provided between the object and image planes.

The optical system may comprises, when in use, an optical element between the detector and the surface to be measured.

The optical element may be a refractive optical element. The optical element may be a lens. The optical element may be a diffractive optical element. The optical element may be reflective.

According to a fifth aspect of the present invention, there is provided a confocal measurement apparatus comprising the optical measurement apparatus as set forth above in relation to the fourth aspect of the invention.

A physiological body-part may comprise the location to be measured. The physiological body-part may be an eye.

A waveguide arrangement may comprise at least part of the optical system. The waveguide arrangement may be a fibre-optic arrangement.

According to a sixth aspect of the present invention, there is provided a method of optically measuring a location to be measured, the method comprising: emitting a probe beam; directing the probe beam to the location to be measured; using an optical system having a detector to receive a reflected beam from the location to be measured, the optical system having a receiving axis; and applying a magnification ratio between the detector and the location to be measured so as to minimise deviation of the reflected beam from the receiving axis.

According to a seventh aspect of the present invention, there is provided a computer program element comprising computer program code means to make a computer execute the method as set forth above in relation to the third or the sixth aspects of the invention.

The computer program element may be embodied on a computer readable medium.

It is thus possible to provide an apparatus and method of measurement for a measurement apparatus that is tolerant to misalignment of a location to be measured, for example a surface of an eye, with an optical system of the measurement apparatus. Consequently, the measurement apparatus benefits from improved consistency of results obtained and increased occasions when measurements can be made, resulting in reduced inconvenience to a user of the measurement apparatus irrespective of whether the user is a clinician or a patient.

At least one embodiment of the invention will now be described, by way of example only, with reference to the accompanying drawing, in which:

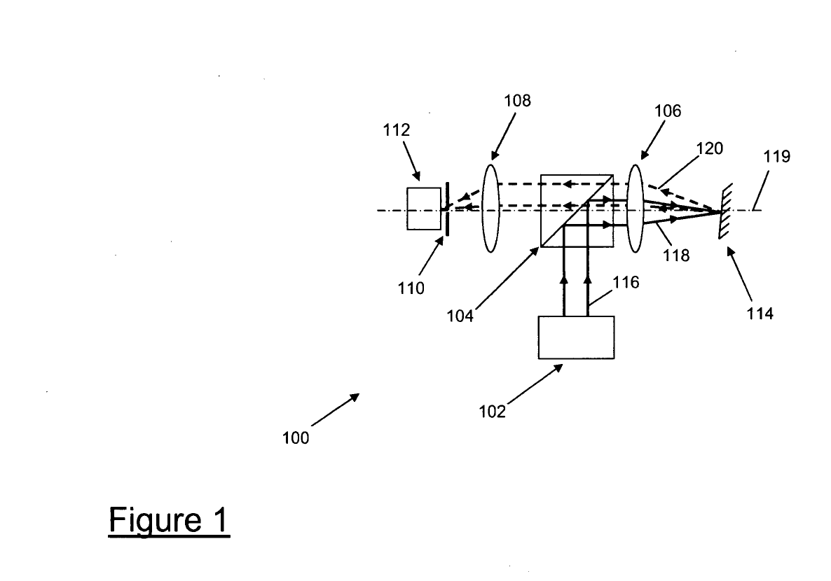

FIG. 1 is a schematic diagram of an apparatus constituting an embodiment of the invention.

Throughout the following description identical reference numerals will be used to identify like parts.

Referring to FIG. 1, an optical measurement apparatus, for example a confocal measurement apparatus, such as a glucometer, comprises an optical system 100 including a source 102 of electromagnetic radiation, for example visible light, though light invisible to the eye, for example near infra-red light, can be used so as to reduce discomfort to the eye. An output window (not shown) of the source 102 is oriented towards a beamsplitter 104 that is placed in an optical path of the source 102. The beamsplitter 104 serves to fold the optical path of the source 102 towards a scanning lens 106. In this example, the scanning lens 106 is shown as a single lens, though the skilled person will appreciate a lens system can serve as the scanning lens 106. The scanning lens 106 is capable of linearly translating so as to move closer to or further from the beamsplitter 104. The skilled person will also understand that the scanning lens 106 can be replaced with a rotating optical element, for example a rotating prism, in order to provide a linear focal point scan. Indeed, a number of design choices are available to the skilled person as alternatives to the scanning lens 106, for example a GRaded INdex (GRIN) lens, a system of mirrors or a diffractive optical element capable of generating a focal spot. Also, non-scanning optical arrangements can be employed, for example as disclosed in UK patent publication no. GB-B-2 407 378, in order to generate a focal line. The beamsplitter 104 is a half-silvered mirror, though the skilled person will appreciate that any suitable alternative optical element or arrangement can be employed to function as the beamsplitter 104, for example a polarising beamsplitter and ¼ wave plate disposed between the polarising beam splitter and the scanning lens 106.

A focussing lens 108 is disposed opposite the beamsplitter 104 so that the beamsplitter 104 is located between the scanning lens 106 and the focussing lens 108. As the optical measurement apparatus is a confocal system, an aperture 110 constituting a pinhole is disposed opposite the focussing lens 108, a detector 112 being disposed adjacent the aperture 110. The detector 112, in this example, comprises a photodiode (not shown in FIG. 1). However, an array of photodiodes or similar detector system, for example Charge-Coupled Device (CCD) detector system or Complementary Metal Oxide Semiconductor (CMOS) detector system, can be employed without the aperture 110 if desired in order to detect off-axis beams. In another embodiment, more than one aperture can be employed, for example of differing diameters in order to de-convolve information about a location to be measured.

The scanning lens 106 has a receiving numerical aperture associated therewith and is capable of accepting light incident thereupon that has a cone angle falling within a receiving angle limit associated with the receiving numerical aperture. Consequently, in this example, the receiving numerical aperture associated with the scanning lens 106 defines an acceptance angle of the optical system 100. An effective numerical aperture of the scanning lens 106 depends upon how the scanning lens 106 is deployed in the optical system 100. For example, a mount for the scanning lens 106 can have a circumferential retaining channel that has side walls that overlie a peripheral annular portion of the scanning lens 106, thereby reducing the usable area of the scanning lens 106.

In operation, the location to be measured is offered to the optical system 100, for example an eye having a surface 114 to be measured. Of course, a surface of the eye is only an example of the many items that can be measured. In this respect, a location in or on any suitable physiological body-part can be measured using the optical system 100. It can now therefore be seen that the optical path mentioned above extends from the source 102 to the detector 112 via the surface 114 to be measured.

After the eye has been brought to the optical system 100, the source 102 emits a beam of collimated light 116, the beam of collimated light 116 being directed to the scanning lens 106. The redirected beam of collimated light is focussed by the scanning lens 106 onto the surface 114 of the eye to be measured. The focussed beam 118, constituting a probe beam, has a probe cone angle corresponding to a probe numerical aperture. The probe beam 118 is incident upon the surface 114, whereupon the probe beam 118 is reflected from the surface 114 at an angle relative to a receiving axis 119 of the optical system 102 depending upon a degree of alignment of the surface 114 with the receiving axis 119, the reflected beam 120 being incident upon the scanning lens 106. In this example, the receiving axis 119 constitutes a substantially ideal optical receive path for receiving light to be processed subsequently.

The reflected beam 120, prior to propagation through the scanning lens 106, also has substantially the probe numerical aperture depending upon the surface profile of the reflecting surface 114. As a result of incidence of the reflected beam 120 on the scanning lens 106, the reflected beam 120 is collimated, the collimated reflected beam 120 propagating through the beam splitter 104 towards the focussing lens 108.

At the focussing lens 108, in accordance with the manner of operation of confocal optical systems, the reflected beam 120 is focussed to the aperture 110 in an image plane of the focussing lens 108. The focussed reflected beam 120 propagates through the aperture 110 and is incident upon the detector 112, the detector 112 having a distribution of light incident over a surface thereof.

In order to ensure substantially full receipt of the reflected beam 120, a number of parameters of the optical system 102 can be adjusted. In this respect, the receiving numerical aperture of the scanning lens 106 can be set to be sufficiently large so as to receive the reflected beam 120 without vignetting. In this example, the receiving numerical aperture is dictated by the physical dimensions of the scanning lens 106. Alternatively or additionally, the probe numerical aperture of the probe beam 118 can be dynamically adaptable, for example dynamically set, in order to ensure that the probe numerical aperture, substantially shared by the reflected beam 120, is of a sufficiently small cone angle to be accepted by the scanning lens 106 without vignetting, i.e. to be within the acceptance angle of the optical system 100.

Consequently, when the reflected beam 120 is off-axis with respect to the receiving axis 119, the reflected beam 120 is still accepted by the scanning lens 106 for propagation as an unattenuated collimated beam towards the focussing lens 108 and hence focussing of the collimated reflected beam 120 onto the aperture 110. The degree of acceptance of off-axis deviation of the reflected beam 120 has a predetermined maximum dictated, in this example, by the receiving numerical aperture associated with the scanning lens 106. Of course, the skilled person will appreciate that the receiving numerical aperture can be constrained by any aperture (not shown) through which the probe beam 118 or the reflected beam 120 (uncollimated) propagates.

In a second embodiment, an object plane exists at the surface 114 and an image plane exists at the aperture 110. In addition, or as an alternative, to manipulation of the probe numerical aperture and/or the receiving numerical aperture, a magnification ratio can be provided between the object and image planes of the optical system 100 in order to mitigate off-axis reflections or deviations at the surface 114 with respect to the receiving axis 119 of the optical system 100. The off-axis reflections or deviations occur, as in relation to the previous embodiment, due to misalignment of the surface 114 to be measured with the receiving axis 119.

In this respect, no additional optical elements are necessarily required between the object and image planes of the optical system 100. In this example, the optical system 100 supports a so-called “angular magnification”. Use of an “optical lever” by virtue of the angular magnification, when correctly configured, for example by relative location of lenses in the optical system 100, results in the magnification ratio and hence a measurement insensitivity to angular displacement of the surface 114.

It should be appreciated that references herein to “light”, other than where expressly stated otherwise, are intended as references relating to the optical range of the electromagnetic spectrum, for example, between about 350 nm and about 2000 nm, such as between about 500 nm and about 1400 nm, or between about 600 nm and about 1000 nm.

In relation to the above embodiments, the skilled person will appreciate that: the selection of the diameters of apertures, for example the diameters of one or more lenses; beam quality, for example intensity profile and/or wavefront profile of the probe beam; and the choice of disposal of an occlusion in the optical path of the probe beam, the reflected beam 120, or both, to alternate on-axis reflections, all influence the “quality” of the optical system 100 and trade-offs between the various parameters on an application-dependent basis need to be made.

It should also be appreciated that the optical system 100 need not be implemented in free space and can be implemented in accordance with a waveguide type design, for example a fibre-optic type design. In this respect, a waveguide arrangement can comprise at least part of the optical system 100.

Although the above examples have predominantly been described in the context of the human eye, the skilled person will appreciate that the techniques described herein can be employed in relation to measurement of any reflecting surface, for example any part of a body, be it human or otherwise. Likewise, measurement of physiological parameters can be made using the above techniques in relation to the body. One example of the physiological parameter is a blood-glucose concentration.

The optical measurement apparatus can be provided as a portable apparatus for personal or clinical use, for example a hand-held device, or table, desk or bench-top apparatus for a clinical environment where a clinician can be present.

1. An optical measurement apparatus comprising:
an optical system comprising:
a source arranged to emit, when in use, a probe beam, the optical system being arranged to direct, when in use, the probe beam to a location to be measured; and
a detector arranged to receive, when in use, a reflected beam from the location to be measured; wherein
the optical system has a receiving numerical aperture associated therewith, the optical system being further arranged so that, when in use, the reflected beam has a cone angle corresponding to a reflected beam numerical aperture; and
the receiving numerical aperture is greater than the reflected beam numerical aperture.

2. An apparatus as claimed in claim 1, wherein the receiving numerical aperture is provided so as to prevent, when in use, vignetting of the reflected beam, the reflected beam numerical aperture being substantially the same as a probe numerical aperture corresponding to a probe cone angle of the probe beam.

3. An apparatus as claimed in claim 1, wherein a probe numerical aperture corresponding to a probe cone angle of the probe beam is set so as to prevent, when in use, vignetting of the reflected beam as a result of the receiving numerical aperture.

4. An apparatus as claimed in claim 1 or claim 2 or claim 3, wherein:
the optical system has a receiving axis associated with the receiving numerical aperture; and
the receiving numerical aperture is arranged to permit acceptance of the reflected beam when deviated from the receiving axis by up to a predetermined maximum off-axis reflection angle with respect to the receiving axis.

5. An apparatus as claimed in claim 1, wherein the optical system is arranged to constrain a probe numerical aperture corresponding to a probe cone angle of the probe beam so that, when in use, the reflected beam is incident within an acceptance angle of the optical system corresponding to the receiving numerical aperture.

6. An apparatus as claimed in any one of the preceding claims, wherein the optical system comprises an optical path from the source to the detector via the location to be measured.

7. An apparatus as claimed in claim 6, wherein the optical system further comprises an optical element disposed in the optical path.

8. An apparatus as claimed in claim 7, wherein the optical element is a refractive optical element.

9. An apparatus as claimed in claim 7, wherein the optical element is an aperture.

10. An apparatus as claimed in claim 7 or claim 8 or claim 9, wherein the optical element provides the receiving numerical aperture.

11. An apparatus as claimed in any one of claims 1 to 10, wherein the probe numerical aperture is dynamically adaptable.

12. An optical measurement apparatus as claimed in claim 1, further comprising a receiving axis, and wherein the optical system is arranged to apply, when in use, a magnification ratio between the detector and the location to be measured so as to minimise deviation of the reflected beam from the receiving axis.

13. An apparatus as claimed in any one of the preceding claims, wherein a waveguide arrangement comprises at least part of the optical system.

14. A confocal measurement apparatus comprising the optical measurement apparatus as claimed in any one of the preceding claims.

15. A method of optically measuring a location to be measured, the method comprising:
emitting a probe beam;
directing the probe beam to the location to be measured;
providing an optical system capable of receiving a reflected beam from the location to be measured, the optical system having a receiving numerical aperture associated therewith; wherein
the reflected beam has a cone angle corresponding to a reflected beam numerical aperture; and
the receiving numerical aperture is greater than the reflected beam numerical aperture.

16. An optical measurement apparatus comprising:
an optical system comprising:
a source arranged to emit, when in use, a probe beam, the optical system being arranged to direct, when in use, the probe beam to a location to be measured;
a detector arranged to receive, when in use, a reflected beam from the location to be measured; and
a receiving axis; wherein
the optical system is arranged to apply, when in use, a magnification ratio between the detector and the location to be measured so as to minimise, when in use, deviation of the reflected beam from the receiving axis.

17. An optical measurement apparatus as claimed in claim 16, wherein the deviation of the reflected beam is due to misalignment of location to be measured with the receiving axis.

18. An apparatus as claimed in claim 16 or claim 17, wherein the optical system has an object plane and an image plane, the magnification ratio being provided between the object and image planes.

19. An apparatus as claimed in any one of claims 16 to 18, wherein the optical system comprises, when in use, an optical element between the detector and the surface to be measured.

20. An apparatus as claimed in claim 19, wherein the optical element is a refractive optical element.

21. A confocal measurement apparatus comprising the optical measurement apparatus as claimed in any one of claims 16 to 20.

22. An apparatus as claimed in any one of claims 1 to 14 or claims 16 to 21, wherein a physiological body-part comprises the location to be measured.

23. An apparatus as claimed in claim 22, wherein the physiological body-part is an eye.

24. An apparatus as claimed in any one of claims 16 to 23, wherein a waveguide arrangement comprises at least part of the optical system.

25. A method of optically measuring a location to be measured, the method comprising:
emitting a probe beam;
directing the probe beam to the location to be measured;
using an optical system having a detector to receive a reflected beam from the location to be measured, the optical system having a receiving axis; and
applying a magnification ratio between the detector and the location to be measured so as to minimise deviation of the reflected beam from the receiving axis.
